(19) United States
(12) Patent Application Publication
(10) Pub. No.: US 2023/0277216 A1
(43) Pub. Date: Sep. 7, 2023

(54) ASSESSMENT DEVICE FOR VULVOVAGINAL REJUVENATION

(71) Applicant: Madorra Inc., Portland, OR (US)

(72) Inventors: Robert BLANTE, Hayward, CA (US); Stephanie KAPLAN, Palo Alto, CA (US); Bryan FLAHERTY, Mountain View, CA (US); Darlene DREON, Portland, OR (US)

(21) Appl. No.: 18/053,588

(22) Filed: Nov. 8, 2022

Related U.S. Application Data
(60) Provisional application No. 63/263,747, filed on Nov. 8, 2021.

(51) Int. Cl. A61B 17/42; A61B 1/303; A61B 1/05; A61B 5/00

(52) U.S. Cl. CPC A61B 17/42; A61B 1/05; A61B 1/303; A61B 5/4337

(57) ABSTRACT

Embodiments of visualization devices for assessing a level of contact between vulvovaginal tissue and a coupling component of a vulvovaginal rejuvenation device are provided. The device can comprise a head portion comprising a window and detachably attached to a coupling pad.

### CROSS REFERENCE TO RELATED APPLICATIONS

This application claims the benefit of U.S. Provisional Pat. Application No. 63/263,747, filed Nov. 8, 2021, the entire disclosure of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This application relates to the field of vulvovaginal rejuvenation.

### BACKGROUND

Vulvovaginal atrophy is a cluster of symptoms associated with decreased estrogen typically found in post-menopausal women. Symptoms commonly include irritation, soreness and post-coital bleeding. Vulvovaginal atrophy also may cause a decrease in lubrication in the vulvovaginal area. As a result, women experiencing vulvovaginal atrophy may not only suffer from decreased sexual enjoyment and day-to-day discomfort due to the lack of lubrication in the vulvovaginal area, but also discomfort during urination and urinary incontinence.

Factors that are known to contribute to vulvovaginal atrophy include menopause, treatments for breast cancer including chemotherapy and for some women, breastfeeding. In all of these causes, a change in the estrogen hormone level is a major contributor to vulvovaginal atrophy.

Until recently, there were limited options for women suffering from vulvovaginal atrophy. Vaginal moisturizers and lubricants only offer temporary relief and often do not provide enough symptomatic relief. Hormone replacement products, either applied locally or systematically, may also be an option, but involve risk of adverse side effects associated with their use. For example, hormone replacement therapies have common side effects such as nausea, vomiting, bloating, weight changes, and in addition may increase the user’s risk of certain cancers and cardiovascular events. Furthermore, these types of hormone-based treatments are not recommended for women with, or who are survivors of, breast, ovarian, or endometrial cancers, and are contraindicated for women with a history of stroke or myocardial infarction because of these risks.

Devices such as those disclosed in U.S. Pat. Application No. 15/767,286, filed Oct. 14, 2016, the entire disclosure of which is incorporated by reference herein, allow for non-invasive vulvovaginal rejuvenation using ultrasound therapy. The device applies ultrasound therapy through a coupling pad engaged with tissue in or around the vagina. Ultrasound treatment devices such as those described therein depend upon satisfactory contact between the device treatment surface and the target tissue. Patient-specific attributes such as anatomy and patient placement can affect the quality of the contact. What is needed is a device to assess the contact and placement of ultrasound treatment devices with respect to the target tissue.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1D show various views of an embodiment of a visualization device.

FIGS. 2A-2C show an embodiment of a coupling pad for use with a visualization device.

FIGS. 3A and 3B show various views of an embodiment of a lens for use in a visualization device.

FIGS. 4A and 4B show various views of an embodiment of a component of a visualization device.

FIGS. 5A and 5B show various views of an embodiment of a vulvovaginal rejuvenation device.

FIGS. 6A and 6B show various views of an embodiment of a visualization device.

### DETAILED DESCRIPTION

The invention described herein provides devices and methods that may be used to assess engagement of a vulvovaginal rejuvenation device with tissue of a women’s vulvovaginal area. The term “vulvovaginal rejuvenation” used herein refers to improving the overall function of the vulvovaginal area that may have suffered from decrease in lubrication, loss of elasticity and resilience, and/or decreased blood flow. Thus, vulvovaginal rejuvenation can refer to any one or a combination of alleviating vaginal dryness, increasing vaginal lubrication, increasing elasticity and/or resilience, and increasing blood flow. Vulvovaginal rejuvenation devices can function by engaging a treatment portion of the device with vaginal tissue and applying energy through the treatment portion to the engaged tissue. In such devices, the quality of engagement between the device and the tissue significantly impacts the efficacy of the treatment. The ability to assess the engagement can enhance treatment, for example, by allowing for determination of patient candidates, adjustment of user placement to accommodate particular anatomy, and correction of incorrect user placement.

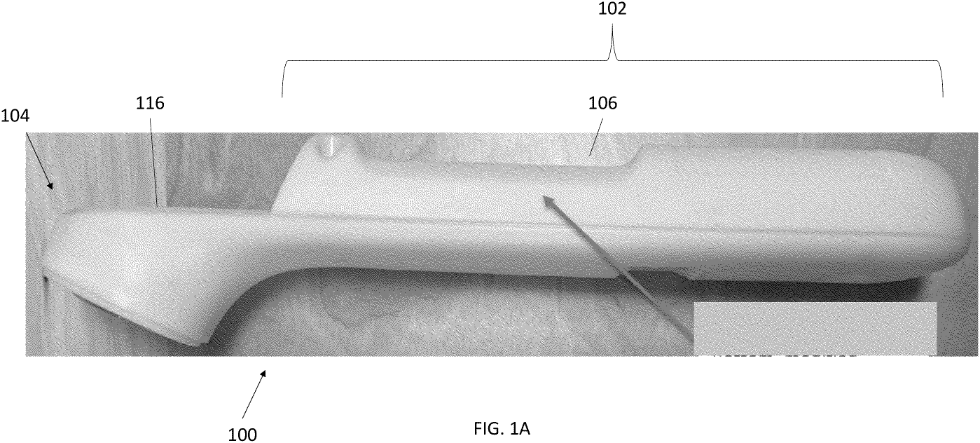

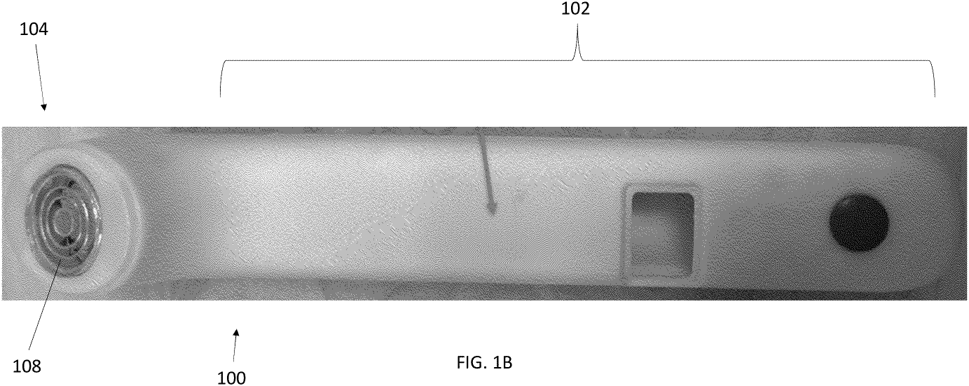

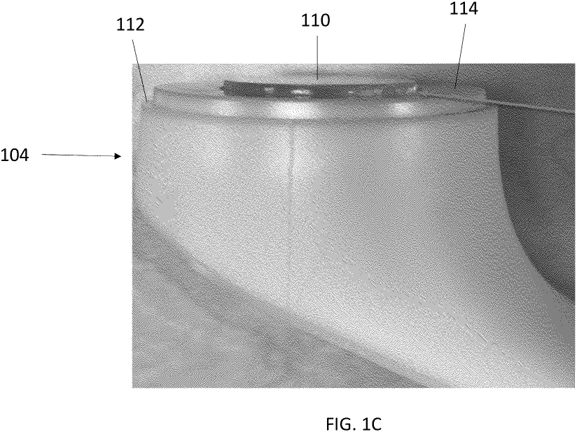

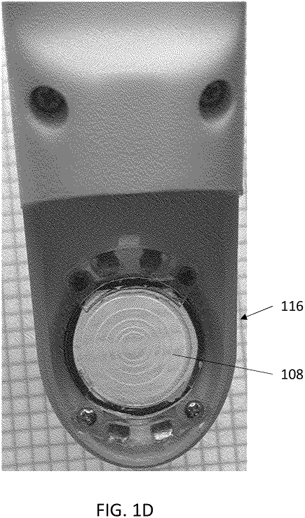

FIGS. 1A and 1B show a side view and a top view, respectively, of an embodiment of a visualization device 100. The device 100 comprises a handle portion 102 and a head portion 104.

The handle portion 102 can comprise a cutout 106 that is designed to aid in a user’s grip of the device. This cutout can greatly improve user ease in holding the device and positioning it properly.

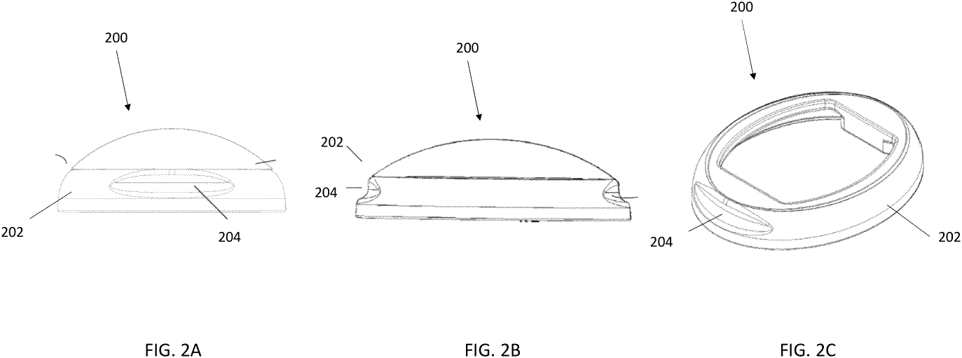

The head portion comprises a visualization window 108. The visualization window can allow for visualization of tissue engaged with a coupling pad (e.g., as shown in FIGS. 2A-2C) attached to the device. The visualization window can comprise a lens, as described in further detail below. In some embodiments, the visualization window comprises an aperture.

The visualization window can comprise a size of about 3-7 cm², 4-6 cm², 5 cm². In some embodiments, the surface area is about 4.9 cm².

In some embodiments, the visualization window is at least as large as a base portion of the coupling pad (e.g., the back surface of the coupling pad that attaches to the device 100). In some embodiments, the visualization window is about the size of the base portion of the coupling pad. In some embodiments, the visualization window is about the size of a tissue engaging portion of the coupling pad.

A back portion 116 of the head portion 102 is open, allowing viewing of the visualization window 108 through the back portion 116, as shown in FIG. 1D.

The head portion 104 comprises attachment means for connecting to the coupling pad portion. The attachment means can comprise magnets on the head portion 104 configured to engage with metal slugs on the coupling pad portion. Magnet attachment means can be easy to use and easy to clean as they may have a low profile. Other attachment means (e.g., hook and loop, snaps, straps, etc.) are also possible. For example, in some embodiments, the attachment means comprises a threaded connection between the head portion and the coupling pad portion, as described elsewhere herein.

The device 100 can comprise plastic (e.g., Polyethylene, Polypropylene, Polystyrene, Polyester, Polycarbonate, Polyvinyl Chloride, Polymethylmethacrylate (PMMA), Polyetheretherketone (PEEK), etc.) with or without a silicone overmold, over certain portions of the handle portion for ease and comfort during use.

The device can have a length of about 200-300 mm, 225-275 mm, about 200 mm, 210 mm, 220 mm, 230 mm, 240 mm, or 250 mm. The device can have a width of about 20-60 mm, 30-40 mm, or about 37 mm. The device handle can have a thickness of about 20-60 mm, 30-40 mm or about 33 mm.

FIG. 1C shows a side view of the head portion. A lens 110 is shown within the visualization window, extending above a top surface 114 of the head portion 104. The head portion 102 comprises a shoulder 112 extending around the top surface 114. A support ring of the coupling pad can be configured to engage with the shoulder 112.

In some embodiments, the head portion of the device comprises lighting configured to illuminate the visualization window and tissue being viewed therethrough.

FIGS. 2A, 2B, and 2C show front, side, and perspective views, respectively, of a coupling pad 200. The coupling pad 200 can have a convex, rounded, dome shape. The shape of the coupling pad can allow the user to self-navigate the device to the proper position near the vagina, at the introitus. In some embodiments, the user navigates to the proper positioning based solely on touch; thus, intuitive positioning can be useful for ensuring proper device use. Other coupling pad configurations are also possible (e.g., taller or shorter dome, central nub, flat top dome, ridge, etc.)

FIGS. 2A-2C show an embodiment of a coupling pad comprising a translucent material. It will be appreciated that other materials (e.g., with different opacity) are also contemplated. It will be further appreciated that the translucency of the coupling pad makes the coupling pad difficult to see in FIG. 2C.

The coupling pad can have a height (above support ring) of about 2-8 mm, about 3-7 mm, about 5-7 mm, or about 6.5 mm.

A support ring 202 of the coupling pad 200 can have depressions 204 on the front and back sides. The depressions can be used to provide ease of handling the coupling pads. A user can place their fingers within the depressions to grip the coupling pad.

The support ring 202 comprises an ovular shape. Other shapes (e.g., circular, rectangular, square, etc.) are also contemplated.

A length of the support ring 202 can be about 30-60 mm, about 40-50 mm, about 45 mm, about 46 mm, or about 47 mm. In some embodiments, the length of the support ring is about 46.6 mm. A width of the support ring can be about 20-50 mm, about 30-40 mm, about 35 mm, about 36 mm, or about 37 mm. In some embodiments, the width of the support ring is about 36 mm.

The opening on a bottom portion of the support ring is filled in by an optically clear coupling pad material and, thus, represents the area of the coupling pad that will engage the device face and visualization window. A large opening in this portion of the support ring enables enhanced visualization of engaged tissue. Additionally, not having such a feature improves manufacturability as injection molding can be used and material cost decreases.

The support ring can comprise a plastic material (e.g., HDPE, Polyethylene, Polypropylene, Polystyrene, Polyester, Polycarbonate, Polyvinyl Chloride, Polymethylmethacrylate (PMMA), Polyetheretherketone (PEEK), etc.).

The bottom surface of the support ring can be configured to mate with the head portion of the main device. The bottom surface comprises attachment means, such as a metal slug or magnets, configured to engage attachment means of the head portion of the main device. As noted above, other attachment means, such as a threaded connection, are also possible.

As noted above, the bottom surface of the coupling pad is the area that will interface with the visualization window. In some embodiments, a surface area of the coupling pad that interfaces with the visualization window is about 3-7 cm², 4-6 cm², 5 cm². In some embodiments, the surface area is about 4.9 cm².

In some embodiments, the coupling pad material comprises a material that can be configured to conform to the tissue (e.g., the area exterior to the vagina, the vulva and introitus). In some embodiments a hydrogel is used. The hydrogel can comprise a combination of any one of agarose, water, Cetylpyridinium chloride (CPC), and glycerin. In some embodiments, the coupling pad comprises about 2% agarose, about 20% glycerin, about 0.5% CPC, and the rest water. Other concentrations are also possible. For example, the coupling pad can comprise about 1-10% agarose. The coupling pad can comprise about 0-25% glycerin. In some embodiments, the coupling pad comprises about 0.5-5% CPC.

In some embodiments, the coupling pad comprises a translucent material which can advantageously enable visualization of the tissue therethrough. In some embodiments, the coupling pad is not completely translucent, but is translucent enough to view the tissue.

As described elsewhere herein, the coupling component can be disposable. In some embodiments, the coupling or disposable component is intended to be disposed of after each assessment or visualization, and a new component is to be used with each assessment or visualization. In other embodiments, a coupling component can be reused between assessments.

In some embodiments, the coupling pad can comprise a durable and stable material (e.g., silicone, acrylic, polyurethane, etc.). In some embodiments, such materials can be used in reusable coupling components for the purposes of visualization.

A magnetic connection between the coupling pad and main device achieves a secure mechanical connection (e.g., defined as a connection force of about 200-600 grams or 300-500 grams) between the device and the face of the coupling pad, with minimal effort required from the user. This can be especially beneficial for users of impaired, deteriorated or otherwise limited dexterity (e.g., those having arthritis, etc.).

The coupling pad 200 used with the visualization device 100 can advantageously be the same coupling pad 200 used with the treatment device. This allows for precise visualization of the coupling pad tissue engagement under simulated use conditions.

Using the treatment coupling pad can also advantageously allow for adjustment or optimization of positioning or placement of the device to accommodate specific anatomy of the user.

In some embodiments, the coupling pad is not the same coupling pad as used by the treatment device. For example, the coupling pad may comprise an outer profile corresponding to or similar to the treatment coupling pad but be hollow instead of solid. In some embodiments, the coupling pad may comprise a different material than the treatment coupling pads.

For another example, the coupling pad may comprise a shape similar to that of the treatment coupling pad. In such embodiments, the coupling pad still provides an assessment of the engagement characteristics of the user’s tissue.

As noted below, in some embodiments, the visualization device shape is similar to the shape of the treatment device. For example, the shape of the handle of the visualization device can be the same or similar to the handle of the treatment device. For example, an angle between the handle and the head portion of the treatment device and the visualization device can be the same or similar. A thickness/diameter of the handle of the treatment device and the visualization device can be the same or similar. This can advantageously allow the visualization device tissue coupling to be the same or similar to the treatment device tissue coupling.

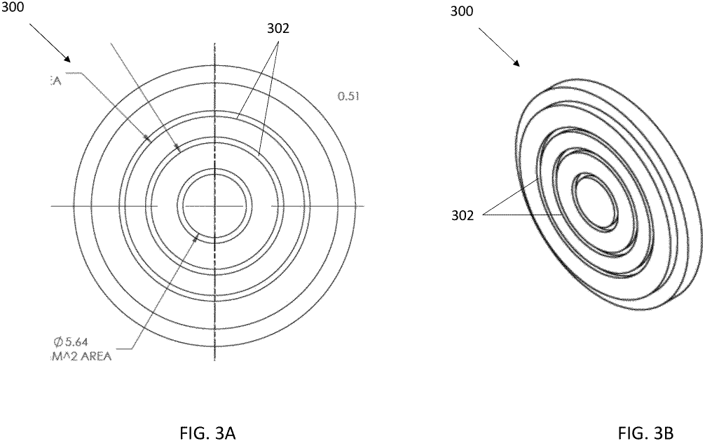

FIGS. 3A and 3B show a perspective view and a top view, respectively of an embodiment of a lens 300 for using over a visualization window of an assessment device (e.g., device 100). In some embodiments, the lens 300 comprises a circular shape. Other configurations (e.g., ovular, square, etc.) are also possible.

In some embodiments, a size of the lens corresponds to a size of an ultrasound device (e.g., ultrasound transducer). This can advantageously provide ease in manufacturing. This can also provide a more accurate assessment of the tissue engagement by providing similar positioning and mechanical forces on the coupling pad.

In some embodiments, the lens comprises features or indicators allowing a user to quantify the size of air bubbles present between the tissue and the coupling pad.

The lens 300 can comprise rings 302 along its surface. In some embodiments, the rings are formed by ridges or grooves in the lens 300 as shown in FIGS. 3A and 3B. Other configurations are also possible (e.g., markings on the lens). In some embodiments, the rings correspond to an enclosed area of about 20-30 mm² (e.g., 25 mm²), about 90-110 mm² (e.g., 100 mm²), and 180-220 mm² (e.g., 200 mm²). In some embodiments, the rings have a diameter of about 5-6 mm (e.g., 5.64 mm), about 10.5-12.5 mm (e.g., 11.28 mm), and 15-17 mm (e.g., 15.95 mm). Other dimensions are also possible.

Rings or other indicators on the lens can also help ensure that the coupling pad is properly centered on the patient anatomy (e.g., over the vaginal canal).

The lens can comprise any rigid, clear material (e.g., PMMA, glass, polycarbonate etc.).

In some embodiments, the lens assembly is formed separately from the device housing. Separate formation can advantageously allow the lens to be polished.

In some embodiments, a coupling pad can be configured to function as the lens. In such embodiments, the device may not have a lens on the visualization window. Instead, the entire coupling pad can comprise an optically polished assembly.

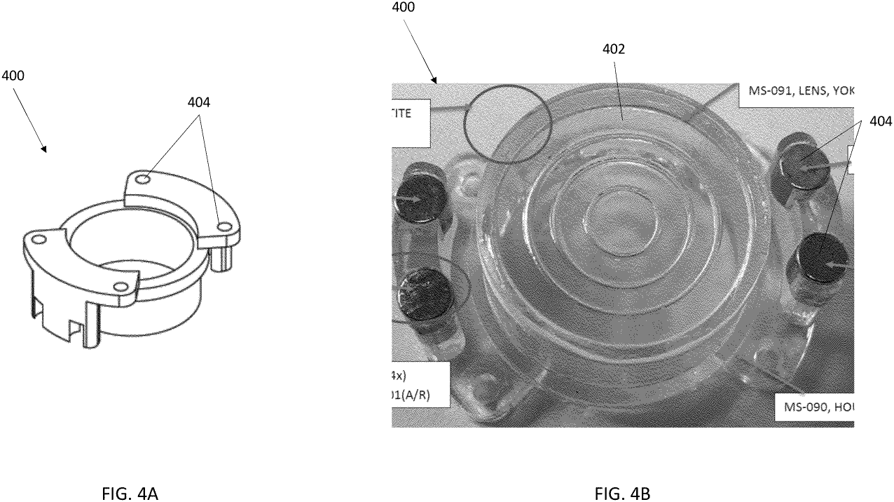

FIGS. 4A and 4B show perspective views of an embodiment of a component 400 of the device housing. The component 400 is configured to receive a lens 402. The component 400 also supports magnets 404 that can be used to magnetically attach a coupling pad to the visualization device.

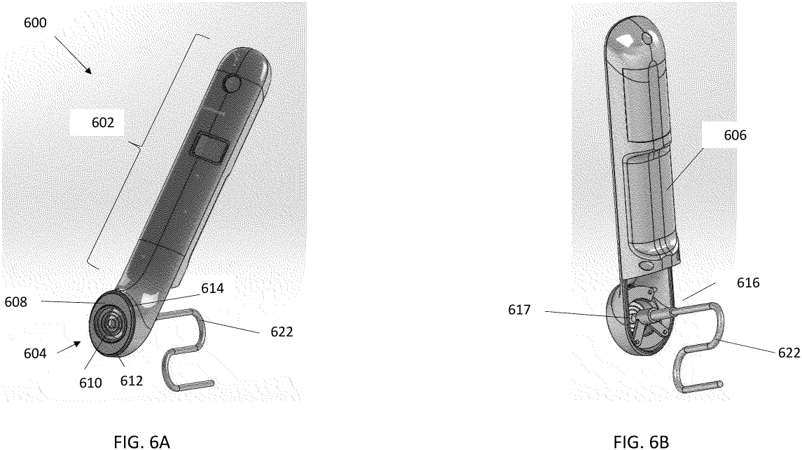

FIGS. 6A and 6B show front and back perspective views of an embodiment of a visualization device 600. Visualization device 600 is similar to visualization device 100, described with respect to FIGS. 1A-1D; however, visualization device 600 comprises a camera configured to allow visualization and assessment of engagement of a vulvovaginal rejuvenation device with tissue of a women’s vulvovaginal area.

The device 600 comprises a handle portion 602 and a head portion 604.

The handle portion 602 can comprise a cutout 606 that is designed to aid in a user’s grip of the device. This cutout can greatly improve user ease in holding the device and positioning it properly.

The head portion can comprise a visualization window 608. The visualization window can allow for visualization of tissue engaged with a coupling pad (e.g., as shown in FIGS. 2A-2C) attached to the device. The visualization window can comprise a lens, as described in further detail below. In some embodiments, the visualization window comprises an aperture. In some embodiments, the camera can be integrated into the window (e.g., such that the front lens of the camera is in contact with the coupling pad).

The visualization window can comprise a size of about 3-7 cm², 4-6 cm², 5 cm². In some embodiments, the surface area is about 4.9 cm².

In some embodiments, the visualization window is at least as large as a base portion of the coupling pad (e.g., the back surface of the coupling pad that attaches to the device 600). In some embodiments, the visualization window is about the size of the base portion of the coupling pad. In some embodiments, the visualization window is about the size of a tissue engaging portion of the coupling pad.

A back portion 616 of the head portion 604 is open, allowing access to the camera 617 and related circuitry through the back portion 616, as shown in FIG. 6B. In some embodiments, the head portion is not open. For example, the device can comprise an enclosed handle with an integrated camera.

The camera 617 can be mounted in a back portion 616 of the head portion 604. In some embodiments, the camera 617 is mounted to the same component to which the lens 608 is mounted (e.g., as described in further detail with respect to FIGS. 4A and 4B).

In some embodiments, the camera 617 is mounted such that the visualization window is within the field of view of the camera 617. In some embodiments, the whole visualization window is not within the field of view of the camera 617, but the camera 617 can be moved or manipulated to allow viewing of the area of interest (e.g., the visualization window).

In some embodiments, the head portion 604 comprises attachment means for connecting to the coupling pad portion. The attachment means can comprise magnets on the head portion 604 configured to engage with metal slugs on the coupling pad portion. Magnet attachment means can be easy to use and easy to clean as they may have a low profile. Other attachment means (e.g., snap fit, hook and loop, snaps, straps, etc.) are also possible. For example, in some embodiments, the attachment means comprises a threaded connection between the head portion and the coupling pad portion, as described elsewhere herein.

The device 600 can comprise plastic (e.g., Polyethylene, Polypropylene, Polystyrene, Polyester, Polycarbonate, Polyvinyl Chloride, Polymethylmethacrylate (PMMA), Polyetheretherketone (PEEK), etc.) with or without a silicone overmold, over certain portions of the handle portion for ease and comfort during use.

The device can have a length of about 200-300 mm, 225-275 mm, about 200 mm, 210 mm, 220 mm, 230 mm, 240 mm, or 250 mm. The device can have a width of about 20-60 mm, 30-40 mm, or about 37 mm. The device handle can have a thickness of about 20-60 mm, 30-40 mm or about 33 mm.

Similar to device 100, a lens 610 is shown within the visualization window, extending above a top surface 614 of the head portion 604. The head portion 604 comprises a shoulder 612 extending around the top surface 614. A support ring of the coupling pad can be configured to engage with the shoulder 612.

In some embodiments of devices with a camera, the lens does not include features or indicators allowing a user to quantify the size of air bubbles present between the tissue and the coupling pad. Instead, the user interface or display configured to show the image captured by the camera may be configured to display features indicators allowing a user to quantify the size of air bubbles present between the tissue and the coupling pad.

In some embodiments, the data from the camera may be analyzed by an associated processor configured to quantify or otherwise assess the level of tissue engagement between the coupling pad and the user’s tissue.

In some embodiments, the image data from the camera is sent to a display associated with the device (e.g., on a computer, tablet, etc.). A user assessing the quality of engagement between the coupling pad and the tissue can view the image on the display to complete the assessment.

In some embodiments, a connector 622 can be used to connect the camera to a display device. In some embodiments, a wireless connection can be used.

In some embodiments, the head portion of the device comprises lighting configured to illuminate the visualization window and tissue being viewed therethrough.

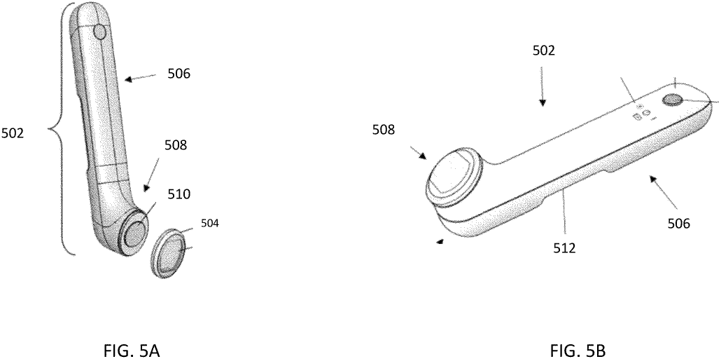

FIGS. 5A and 5B show an embodiment of an ultrasound device 500 with which the visualization devices described herein (e.g., device 100) may be used. The device 500 comprises a main device 502 and a coupling pad 504 (e.g., pad 200).

FIG. 5B shows a perspective view of the device 500 without the coupling component attached. The main device 502 comprises a handle portion 506 and a head portion 508. In some embodiments, the head portion 508 of the device 500 comprises an ultrasound transducer 510. The ultrasound transducer can be a flat, disc-type transducer. Other configurations (e.g., curved) are also possible. In some embodiments, the ultrasound transducer is a ceramic piezoelectric crystal. Other transducers are also possible. The transducer can have a diameter of about 15-25 mm, about 15 mm, about 20 mm, or about 25 mm. Other transducer sizes are also contemplated. The effective radiating area (Aer) of the transducer can be about 2 cm². Other areas are also possible (e.g., 2-12 cm², 2-8 cm², 2-6 cm², 2-4 cm², or about 3 cm²).

The head portion 508 also comprises attachment means for connecting to the coupling pad portion. The attachment means can comprise magnets on the head portion 508 configured to engage with magnets on the coupling pad portion. Other attachment means (e.g., hook and loop, snaps, straps, etc.) are also possible. For example, in some embodiments, the attachment means comprises a threaded connection between the head portion and the coupling pad portion, as described elsewhere herein.

The handle portion can comprise a cutout 512 that is designed to aid in a user’s grip of the device. User testing has shown that this cutout greatly improves user ease in holding the device and positioning it properly.

Similar to the visualization device 100, the device can have a length of about 200-300 mm, 225-275 mm, about 200 mm, 210 mm, 220 mm, 230 mm, 240 mm, or 250 mm. The device can have a width of about 20-60 mm, 30-40 mm, or about 37 mm. The device handle can have a thickness of about 20-60 mm, 30-40 mm or about 33 mm.

In some embodiments, the same device shell is used for the treatment device and the visualization device. Using the same device shell may advantageously allow for accurate recreation of the therapy and subsequent diagnosis of an issue with tissue engagement.

To use a visualization device as described herein (e.g., device 100) to assess engagement of a coupling pad with a user’s vulvovaginal tissue, a clinician or user can attach a coupling pad to the head portion of the visualization device. The clinician or user can engage the coupling pad with tissue in the user’s vulvovaginal area, for example, at the vulva and introitus. The clinician may view the visualization window from the back side of the head portion of the device to assess the tissue engagement.

If an air bubble is observed, the clinician may use markings (e.g., rings) on the lens of the visualization window to quantify the size of the air bubble(s).

In some embodiments, air bubbles above a threshold size may indicate that a particular user is not a good candidate for the vulvovaginal rejuvenation device. In certain such users, vaginal laxity of a certain degree may lead to this result.

In some embodiments, observation of air bubbles may lead the clinician to adjust the position or placement (e.g., pressure, back angle, knee position, etc.) of the device against the vulvovaginal tissue.

Correcting improper placement or positioning may alleviate the presence of air bubbles. For example, the user can position the device, performing a mock therapy. Based on the clinician’s observation of air bubbles, the placement may be adjusted to reduce or minimize the formation of air bubbles.

In some embodiments adjusting the placement or positioning can optimize positioning or help alleviate the presence of air bubbles for users that are using the device correctly. For example, the user can position the device, performing a mock therapy. Based on the clinician’s observation of air bubbles, the clinician and user may adapt the placement of the device to a position at which the formation of air bubbles is reduced or minimized.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature’s relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/- 0.1% of the stated value (or range of values), +/- 1% of the stated value (or range of values), +/- 2% of the stated value (or range of values), +/- 5% of the stated value (or range of values), +/- 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A handheld device for visualizing quality of contact between a vulvovaginal rejuvenation device and the introitus, comprising:
a handle configured for maintaining a position of the device during use;
a head portion comprising a translucent visualization window; and
a coupling pad configured to be attached to the head portion, the coupling pad configured to conform to tissue in or around a user’s vagina and external genitalia.

2. The device of claim 1, wherein the visualization window comprises a lens.

3. The device of claim 2, wherein the lens comprises markings or indicators allowing measurement of features seen therethrough.

4. The device of claim 1, the device further comprising a light configured to illuminate the visualization window.

5. The device of claim 1, wherein the coupling pad is detachably attached to the head portion.

6. The device of claim 1, wherein the device comprises a camera configured to provide image data captured through the visualization window.

7. The device of claim 6, wherein the camera is configured to send image data to a display associated with the device.

8. The device of claim 1, wherein the coupling pad comprises a height of about 3-7 mm.

9. The device of claim 1, wherein the coupling pad comprises a generally ovular shape.

10. The device of claim 1, wherein the coupling pad comprises a generally dome shaped configuration.

11. The device of claim 1, wherein the visualization window comprises a surface area of about 3-7 cm².

12. The device of claim 1, wherein the coupling pad is configured to be attached to the head portion via snap fit.

13. The device of claim 1, wherein the coupling pad is configured to be attached to the head portion via magnetic connection.

14. The device of claim 1, wherein the coupling pad comprises a support ring.

15. The device of claim 1, wherein the coupling pad comprises a translucent material.

16. A method for assessing placement of a vulvovaginal rejuvenation device, comprising
using a handle to position a coupling pad of a head portion of the device against a user’s vulvovaginal tissue; and
observing engagement of the vulvovaginal tissue and the coupling pad through a visualization window in a head portion of the visualization device.

17. The method of claim 16, wherein observing engagement of the vulvovaginal tissue and the coupling pad through the visualization window comprises viewing the engagement through a lens positioned on the visualization window.

18. The method of claim 16, further comprising adjusting the position of the device.

19. The method of claim 16, further comprising measuring a size of an air bubble using markings on the lens or visualization window.

20. The method of claim 16, wherein observing engagement of the vulvovaginal tissue and the coupling pad through a visualization window in a head portion of the visualization device comprises viewing data from a camera.

21. The method of claim 16, further comprising adjusting the position of the device until a size of an observed air bubble decreases.

22. The method of claim 16, further comprising detachably connecting the coupling pad to the head portion of the device.
